(19) United States
(12) Patent Application Publication
(10) Pub. No.: US 2009/0247617 A1
(43) Pub. Date: Oct. 1, 2009

(54) PROCESS FOR THE PREPARATION OF BENZO-FUSED HETEROARYL SULFAMATES

(76) Inventors: Ahmed F. Abdel-Magid, Ambler, PA (US); Steven J. Mehrman, Quakertown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/055,695

(22) Filed: Mar. 26, 2008

(51) Int. Cl. A61K 31/357; C07D 319/16; C07D 405/06

(52) U.S. Cl. 514/452; 549/362; 548/454

(57) ABSTRACT

The present invention is directed to a process for the preparation of benzo-fused heteroaryl sulfamates, useful for the treatment of epilepsy and related disorders.

### FIELD OF THE INVENTION

The present invention is directed to a process for the preparation of benzo-fused heteroaryl sulfamates, useful for the treatment of epilepsy and related disorders.

### BACKGROUND OF THE INVENTION

Epilepsy describes a condition in which a person has recurrent seizures due to a chronic, underlying process. Epilepsy refers to a clinical phenomenon rather than a single disease entity, since there are many forms and causes of epilepsy. Using a definition of epilepsy as two or more unprovoked seizures, the incidence of epilepsy is estimated at approximately 0.3 to 0.5 percent in different populations throughout the world, with the prevalence of epilepsy estimated at 5 to 10 people per 1000.

An essential step in the evaluation and management of a patient with a seizure is to determine the type of seizure that has occurred. The main characteristic that distinguishes the different categories of seizures is whether the seizure activity is partial (synonymous with focal) or generalized.

Partial seizures are those in which the seizure activity is restricted to discrete areas of the cerebral cortex. If consciousness is fully preserved during the seizure, the clinical manifestations are considered relatively simple and the seizure is termed a simple-partial seizure. If consciousness is impaired, the seizure is termed a complex-partial seizure. An important additional subgroup comprises those seizures that begin as partial seizures and then spread diffusely throughout the cortex, which are known as partial seizures with secondary generalization.

Generalized seizures involve diffuse regions of the brain simultaneously in a bilaterally symmetric fashion. Absence or petit mal seizures are characterized by sudden, brief lapses of consciousness without loss of postural control. Atypical absence seizures typically include a longer duration in the lapse of consciousness, less abrupt onset and cessation, and more obvious motor signs that may include focal or lateralizing features. Generalized Tonic-clonic or grand mal seizures, the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 s, the tonic phase of the seizure typically evolves into the clonic phase, produced by the superimposition of periods of muscle relaxation on the tonic muscle contraction. The periods of relaxation progressively increase until the end of the ictal phase, which usually lasts no more than 1 min. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 s. Consciousness is briefly impaired, but there is usually no postictal confusion. Myoclonic seizures are characterized by a sudden and brief muscle contraction that may involve one part of the body or the entire body. (Harrison's Online, Mar. 29, 2001)

McComsey, D., et al. in US Patent Publication US 2006/0041008 A1, published Feb. 23, 2006 and McComsey, D., et al. in US Patent Publication US 2005/0282887 A1, published Dec. 22, 2005 disclose compounds of formula (I) and their use in the treatment of epilepsy and related disorders. McComsey, D., et al. in US Patent Publication US 2006/0041008 A1 and McComsey, D., et al. in US Patent Publication US 2005/0282887 A1 further disclose a process for the preparation of the compounds of formula (I) comprising reacting a suitable substituted amine with sulfamide.

In the process(es) as disclosed in McComsey D., et al. compounds of formula (I) wherein R¹ and R² are each hydrogen describes the use of sulfamoyl chloride (Cl—SO₂—NH₂) as a reagent, which reagent is unsuitable for large scale/commercial preparation. There remains, however, a need for a process suitable for the preparation of large scale material and/or for commercial preparation of the compounds of formula (I).

### SUMMARY OF THE INVENTION

The present invention is directed to processes for the preparation of compounds of formula (I)

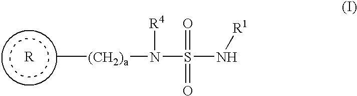

wherein

R¹ is selected from the group consisting of hydrogen and lower alkyl;

R⁴ is selected from the group consisting of hydrogen and lower alkyl;

a is an integer from 1 to 2;

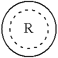

is selected from the group consisting of

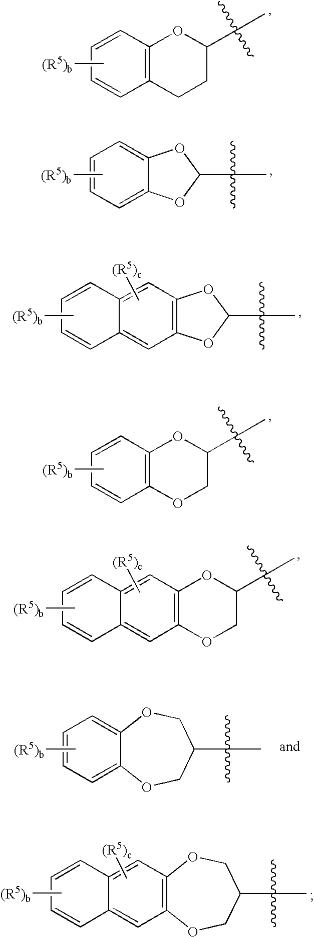

wherein b is an integer from 0 to 4; and wherein c is an integer from 0 to 2;

each R⁵ is independently selected from the group consisting of halogen, lower alkyl and nitro;

provided that when

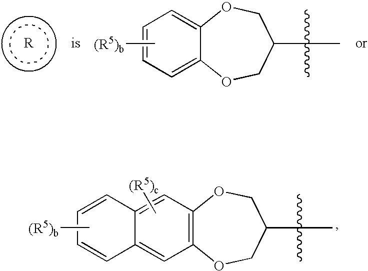

then a is 1;

or a pharmaceutically acceptable salt thereof.

The present invention is directed to a process for the preparation of compounds of formula (IA)

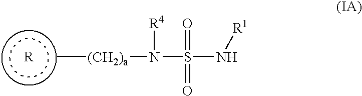

wherein

R¹ is hydrogen;

R⁴ is selected from the group consisting of hydrogen and lower alkyl;

a is an integer from 1 to 2;

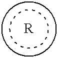

is selected from the group consisting of

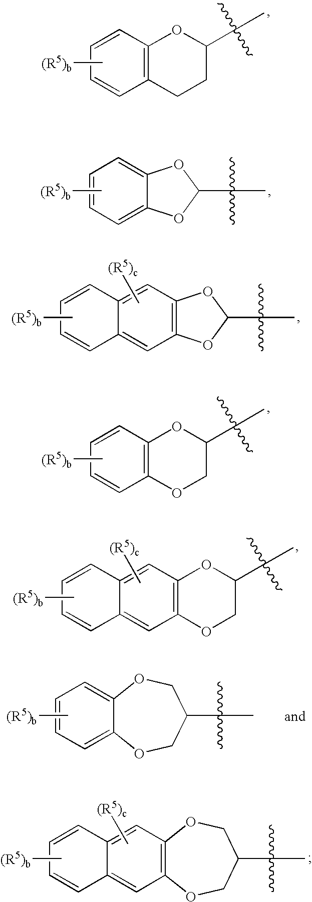

wherein b is an integer from 0 to 4; and wherein c is an integer from 0 to 2;

each R⁵ is independently selected from the group consisting of halogen, lower alkyl and nitro;

provided that when

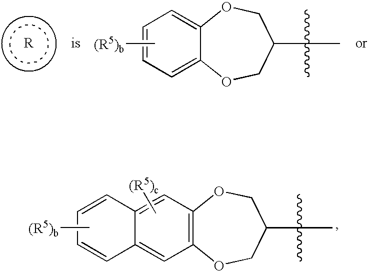

then a is 1;

or a pharmaceutically acceptable salt thereof;

comprising

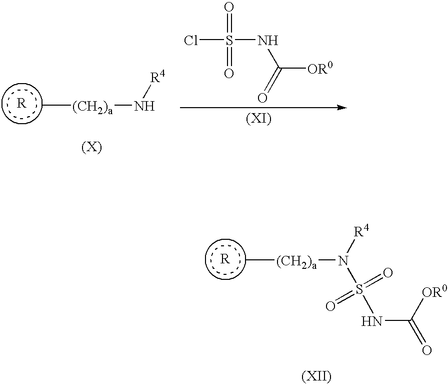

reacting a compound of formula (X) with a compound of formula (XI) wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII);

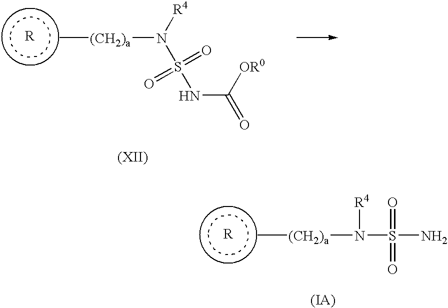

de-protecting the compound of formula (XII); to yield the corresponding compound of formula (IA).

The present invention is further directed to a process for the preparation of a compound of formula (IB)

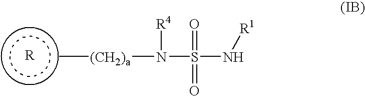

wherein

R¹ is selected from the group consisting of lower alkyl;

R⁴ is selected from the group consisting of hydrogen and lower alkyl;

a is an integer from 1 to 2;

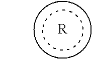

is selected from the group consisting of

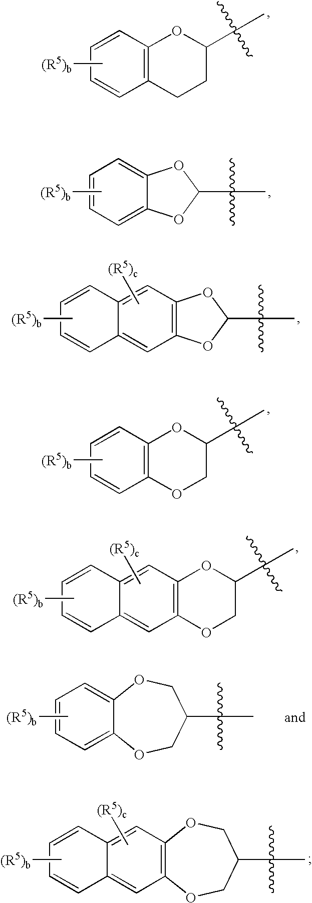

wherein b is an integer from 0 to 4; and wherein c is an integer from 0 to 2;

each R⁵ is independently selected from the group consisting of halogen, lower alkyl and nitro;

provided that when

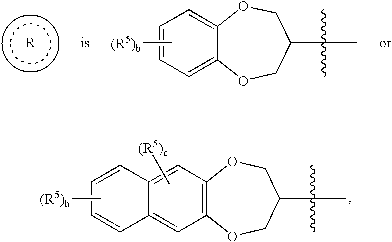

then a is 1;

or a pharmaceutically acceptable salt thereof;

comprising

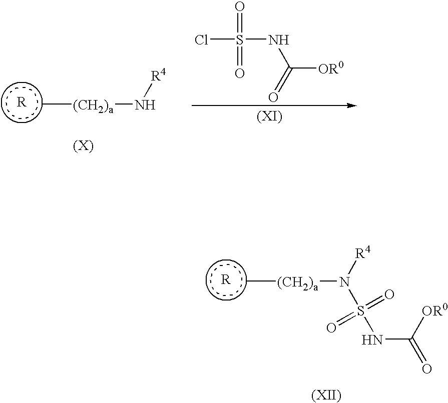

reacting a compound of formula (X) with a compound of formula (XI) wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII);

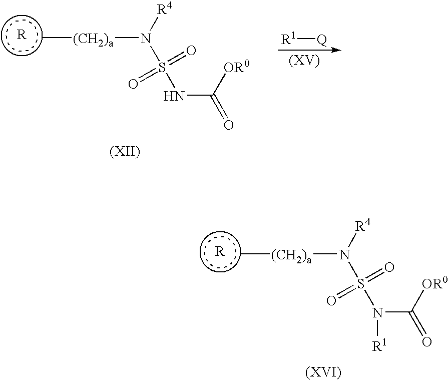

reacting the compound of formula (XII) with a compound of formula (XV), wherein Q is a leaving group; in an organic solvent; to yield the corresponding compound of formula (XVI)

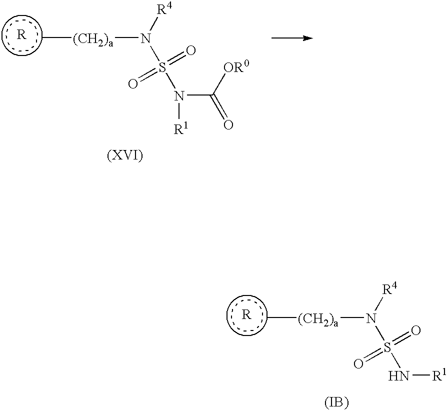

de-protecting the compound of formula (XVI); to yield the corresponding compound of formula (IB).

In an embodiment, the present invention is directed to a process for the preparation of a compound of formula (I-S)

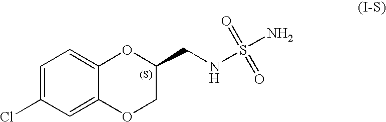

or a pharmaceutically acceptable salt thereof (also known as N-[[(2S)-6-chloro-2,3-dihydro-1,4-benzodioxin-2-yl]methyl]-sulfamide) comprising

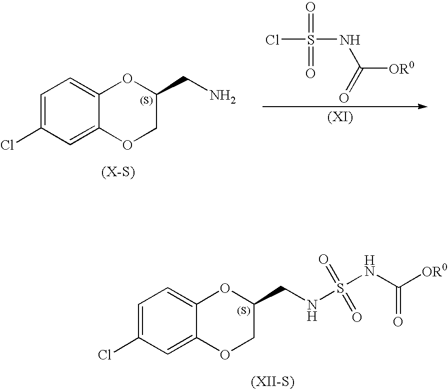

reacting a compound of formula (X-S) with a compound of formula (XI), wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII-S);

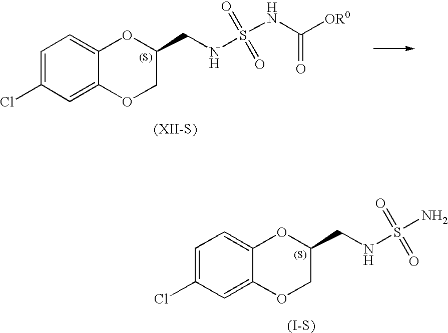

de-protecting the compound of formula (XII-S); to yield the corresponding compound of formula (I-S).

The present invention is further directed to a crystalline form of the compound of formula (I-S), hereinafter referred to as crystalline form (I-SA).

The present invention is further directed to compounds of formula (XII)

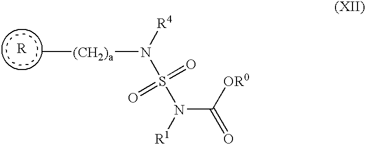

wherein —C(O)OR⁰ is a nitrogen protecting group and wherein

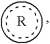

a, R⁴ and R¹ are as herein defined. Preferably, —C(O)OR⁰ is a nitrogen protecting group wherein R⁰ is selected from the group consisting of C₁₋₄alkyl (preferably t-butyl), benzyl, p-methoxybenzyl and 9-fluorenylmethyl, more preferably, —C(O)OR⁰ is tert-butoxycarbonyl (i.e. R⁰ is t-butyl). The compounds of formula (XII) are useful as intermediates in the synthesis of the compounds of formula (I).

The present invention is further directed to a product prepared according to the process described herein.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

Exemplifying the invention are methods of treating epilepsy or a related disorder comprising administering to a subject in need thereof, a therapeutically effective amount of any of the compounds or pharmaceutical compositions described above.

### BRIEF DESCRIPTION OF THE FIGURES

FIG. 1 illustrates a representative XRD Spectra for Crystalline Form of the Compound of Formula (I-S)

### DETAILED DESCRIPTION OF THE INVENTION

The present invention is directed to processes for the preparation of compound of formula (I)

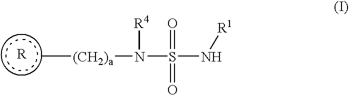

wherein R¹, R⁴, a and

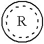

are as herein defined. The compounds of formula (I) are useful in the treatment of epilepsy and related disorders.

In an embodiment, the present invention is directed to a process for the synthesis of compounds of formula (IA)

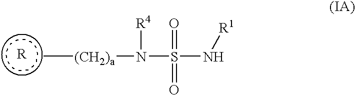

wherein R¹ is hydrogen, and wherein R⁴, a and

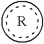

are as herein defined; and pharmaceutically acceptable salts thereof.

In another embodiment, the present invention is directed to a process for the preparation of compounds of formula (IB)

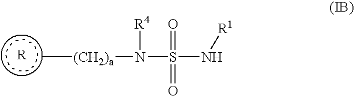

wherein R¹ is selected from the group consisting of lower alkyl, and wherein R⁴, a and

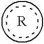

are as herein defined; and pharmaceutically acceptable salts thereof.

In another embodiment, the present invention is directed to a process for the synthesis of compounds of formula (IC)

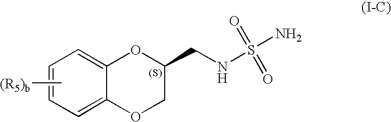

and pharmaceutically acceptable salts thereof, wherein b and R⁵ are as herein defined. Preferably, b is an integer from 0 to 2; more preferably, b is an integer from 0 to 1. Preferably R⁵ is halogen, more preferably chloro.

In an embodiment of the present invention R¹ is selected from the group consisting of hydrogen and methyl. In another embodiment of the present invention, R¹ is hydrogen.

In an embodiment of the present invention —(CH₂)ₐ— is selected from the group consisting of —CH₂— and —CH₂—CH₂—. In another embodiment of the present invention —(CH₂)ₐ— is —CH₂—.

In an embodiment of the present R⁴ is selected from the group consisting of hydrogen and methyl, preferably, R⁴ is hydrogen.

In an embodiment of the present invention a is 1.

In an embodiment of the present invention b is an integer from 0 to 2. In another embodiment of the present invention c is an integer from 0 to 2. In another embodiment of the present invention b is an integer from 0 to 1. In another embodiment of the present invention c is an integer from 0 to 1. In yet another embodiment of the present invention the sum of b and c is an integer from 0 to 2, preferably an integer from 0 to 1. In yet another embodiment of the present invention b is an integer from 0 to 2 and c is 0.

In an embodiment of the present invention,

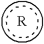

is a ring structure selected from the group consisting of

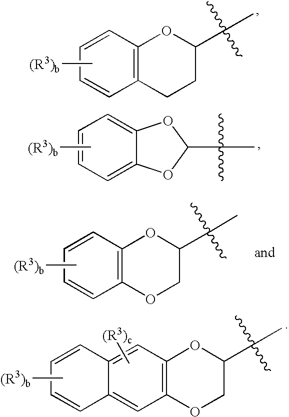

In another embodiment of the present invention,

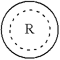

is a ring structure selected from the group consisting of

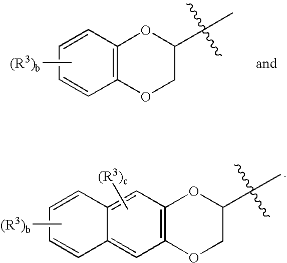

In an embodiment of the present invention,

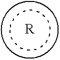

is a ring structure selected from the group consisting of 2-(chromanyl), 2-(6-chloro-2,3-dihydro-benzo[1,4]dioxinyl), 2-(benzo[1,3]dioxolyl), 2-(5-chloro-2,3-dihydro-benzo[1,4]dioxinyl), 2-(7-nitro-2,3-dihydro-benzo[1,4]dioxinyl), 2-(6,7-dichloro-2,3-dihydro-benzo[1,4]dioxinyl), 2-(2,3-dihydro-naphtho[2,3-b][1,4]dioxinyl) and 2-(7-chloro-benzo[1,3]dioxolyl). In another embodiment of the present invention,

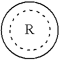

is a ring structure selected from the group consisting of 2-(5-chloro-2,3-dihydro-benzo[1,4]dioxinyl), 2-(7-nitro-2,3-dihydro-benzo[1,4]dioxinyl), 2-(6,7-dichloro-2,3-dihydro-benzo[1,4]dioxinyl) and 2-(2,3-dihydro-naphtho[2,3-b][1,4]dioxinyl).

In an embodiment of the present invention,

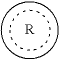

is selected from the group consisting of

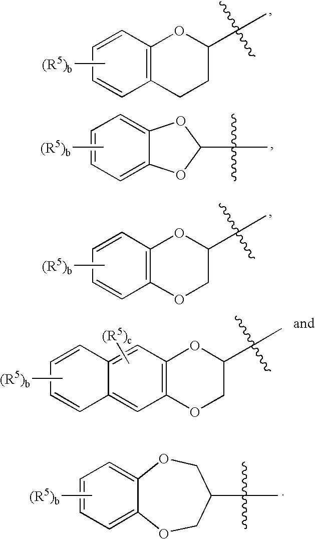

In another embodiment of the present invention,

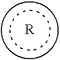

is selected from the group consisting of

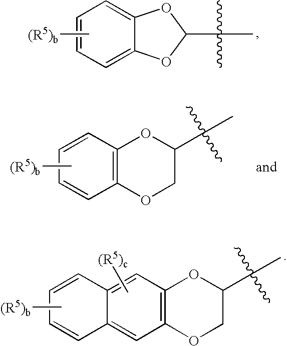

In an embodiment of the present invention,

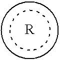

is selected from the group consisting of 2-(2,3-dihydro-benzo[1,4]dioxinyl), 2-(benzo[1,3]dioxolyl), 2-(3,4-dihydro-benzo[1,4]dioxepinyl), 2-(6-chloro-2,3-dihydro-benzo[1,4]dioxinyl), 2-(6-fluoro-2,3-dihydro-benzo[1,4]dioxinyl), 2-(chromanyl), 2-(5-fluoro-2,3-dihydro-benzo[1,4]dioxinyl), 2-(7-chloro-2,3-dihydro-benzo[1,4]dioxinyl), 2-(6-chloro-benzo[1,3]dioxolyl), 2-(7-nitro-2,3-dihydro-benzo[1,4]dioxinyl), 2-(7-methyl-2,3-dihydro-benzo[1,4]dioxinyl), 2-(5-chloro-2,3-dihydro-benzo[1,4]dioxinyl), 2-(6-bromo-2,3-dihydro-benzo[1,4]dioxinyl), 2-(6,7-dichloro-2,3-dihydro-benzo[1,4]dioxinyl), 2-(8-chloro-2,3-dihydro-benzo[1,4]dioxinyl), 2-(2,3-dihydro-naphtho[2,3-b][1,4]dioxinyl) and 2-(4-methyl-benzo[1,3]dioxolyl).

In another embodiment of the present invention,

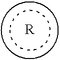

is selected from the group consisting 2-(benzo[1,3]dioxolyl), 2-(2,3-dihydro-benzo[1,4]dioxinyl), 2-(6-chloro-2,3-dihydro-benzo[1,4]dioxinyl), 2-(7-chloro-2,3-dihydro-benzo[1,4]dioxinyl), 2-(7-methyl-2,3-dihydro-benzo[1,4]dioxinyl), 2-(6-bromo-2,3-dihydro-benzo[1,4]dioxinyl) and 2-(6,7-dichloro-2,3-dihydro-benzo[1,4]dioxinyl). In another embodiment of the present invention,

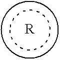

is selected from the group consisting of 2-(2,3-dihydro-benzo[1,4]dioxinyl), 2-(7-methyl-2,3-dihydro-benzo[1,4]dioxinyl) and 2-(6-bromo-2,3-dihydro-benzo[1,4]dioxinyl).

In an embodiment of the present invention R³ is selected from the group consisting of halogen, lower alkyl, hydroxy substituted lower alkyl, —O-(lower alkyl), nitro, cyano, amino, lower alkylamino and di(lower alkyl)amino. In another embodiment of the present invention R³ is selected from the group consisting of halogen and nitro. In another embodiment of the present invention R³ is selected from the group consisting of chloro and nitro.

In an embodiment of the present invention R⁵ is selected from the group consisting of (II) halogen and lower alkyl. In another embodiment of the present invention R⁵ is selected from chloro, fluoro, bromo and methyl.

In an embodiment of the present invention, in the compound of formula (I),

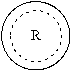

is other than

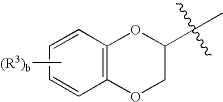

wherein b is 1 and R³ is selected from the group consisting of halogen, nitro, cyano, amino, lower alkyl, lower alkoxy and —C(O)O-(lower alkyl). In another embodiment of the present invention, in the compound of formula (I),

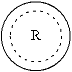

is other than

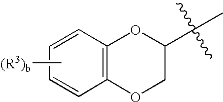

wherein b is 1.

In an embodiment of the present invention, the stereo-center on the compound of formula (I) is in the S-configuration. In another embodiment of the present invention, the stereo-center on the compound of formula (I) is in the R-configuration.

In an embodiment of the present invention the compound of formula (I) is present as an enantiomerically enriched mixture, wherein the % enantiomeric enrichment (% ee) is greater than about 75%, preferably greater than about 85%, more preferably greater than about 90%, more preferably greater than about 95%, most preferably greater than about 98%.

In another embodiment, the present invention is directed to one or more of the representative compounds of formula (I) as listed in Table 1, below. In Table 1 below, the column headed “stereo” defines the stereo-configuration at the carbon atom of the heterocycle attached at the starred bond. Where no designation is listed, the compound was prepared as a mixture of stereo-configurations. Where an “R” or “S” designation is listed, the stereo-configuration was based on the enantiomerically enriched starting material.

TABLE 1
Representative Compounds of Formula (I)

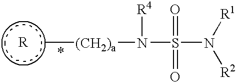

| ID No. | 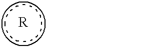 | Stereo | (CH₂)ₐ | NR⁴ | R¹ | R² |
|---|---|---|---|---|---|---|
| 1 | 2-(2,3-dihydro- | | CH₂ | NH | H | H |
| | benzo[1,4]dioxinyl) | | | | | |
| 2 | 2-(benzo[1,3]dioxolyl) | | CH₂ | NH | H | H |
| 3 | 2-(3,4-dihydro-2H- | | CH₂ | NH | H | H |
| | benzo[1,4]dioxepinyl) | | | | | |
| 4 | 2-(2,3-dihydro- | S | CH₂ | NH | H | H |
| | benzo[1,4]dioxinyl) | | | | | |
| 5 | 2-(2,3-dihydro- | R | CH₂ | NH | H | H |
| | benzo[1,4]dioxinyl) | | | | | |
| 7 | 2-(2,3-dihydro- | | CH₂ | N(CH₃) | H | H |
| | benzo[1,4]dioxinyl) | | | | | |
| 8 | 2-(6-chloro-2,3-dihydro- | S | CH₂ | NH | H | H |
| | benzo[1,4]dioxinyl) | | | | | |
| 9 | 2-(6-fluoro-2,3-dihydro- | S | CH₂ | NH | H | H |
| | benzo[1,4]dioxinyl) | | | | | |
| 10 | 2-(chromanyl) | | CH₂ | NH | H | H |
| 13 | 2-(5-fluoro-2,3-dihydro- | S | CH₂ | NH | H | H |
| | benzo[1,4]dioxinyl) | | | | | |
| 14 | 2-(7-chloro-2,3-dihydro- | S | CH₂ | NH | H | H |
| | benzo[1,4]dioxinyl) | | | | | |
| 15 | 2-(6-chloro- | | CH₂ | NH | H | H |
| | benzo[1,3]dioxolyl) | | | | | |
| 16 | 2-(2,3-dihydro- | | CH₂CH₂ | NH | H | H |
| | benzo[1,4]dioxinyl) | | | | | |
| 18 | 2-(7-nitro-2,3-dihydro- | S | CH₂ | NH | H | H |
| | benzo[1,4]dioxinyl) | | | | | |
| 19 | 2-(7-methyl-2,3-dihydro- | S | CH₂ | NH | H | H |
| | benzo[1,4]dioxinyl) | | | | | |
| 20 | 2-(5-chloro-2,3-dihydro- | S | CH₂ | NH | H | H |
| | benzo[1,4]dioxinyl) | | | | | |
| 22 | 2-(8-methoxy-2,3- | S | CH₂ | NH | H | H |
| | dihydro- | | | | | |
| | benzo[1,4]dioxinyl) | | | | | |
| 24 | 2-(6-bromo-2,3-dihydro- | S | CH₂ | NH | H | H |
| | benzo[1,4]dioxinyl) | | | | | |
| 29 | 2-(6,7-dichloro-2,3- | S | CH₂ | NH | H | H |
| | dihydro- | | | | | |
| | benzo[1,4]dioxinyl) | | | | | |
| 30 | 2-(8-chloro-2,3-dihydro- | S | CH₂ | NH | H | H |
| | benzo[1,4]dioxinyl) | | | | | |
| 33 | 2-(2,3-dihydro- | S | CH₂ | NH | H | H |
| | naphtho[2,3- | | | | | |
| | b][1,4]dioxinyl) | | | | | |
| 35 | 2-(4-methyl- | | CH₂ | NH | H | H |
| | benzo[1,3]dioxolyl) | | | | | |

Additional embodiments of the present invention, include those wherein the substituents selected for one or more of the variables defined herein (e.g. a, R¹, R⁴, R ⁵,

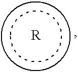

etc.) are independently selected to be any individual substituent or any subset of substituents selected from the complete list as defined herein.

The present invention is further directed to compounds of formula (XII)

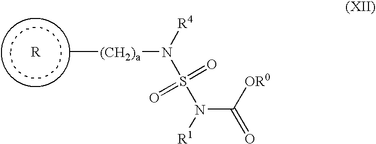

wherein —C(O)OR⁰ is a nitrogen protecting group and wherein

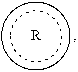

a, R⁴ and R¹ are as herein defined. In an embodiment, the present invention is directed to a compound of formula (XII-S)

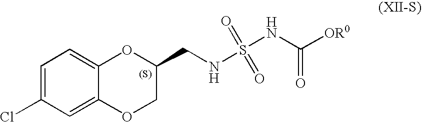

wherein —C(O)OR⁰ is a nitrogen protecting group. Preferably, —C(O)OR⁰ is a nitrogen protecting group wherein R⁰ is selected from the group consisting of alkoxycarbonyl, aryloxycarbonyl, aralkyloxycarbonyl. More preferably, R⁰ is selected from the group consisting of C₁₋₄alkyl (preferably t-butyl), benzyl, p-methoxybenzyl, phenylethyl, phenyl, naphthyl, cycloalkyl and 9-fluorenylmethyl. More preferably, R⁰ is selected from the group consisting of C₁₋₄alkyl (preferably t-butyl), benzyl, p-methoxybenzyl and 9-fluorenylmethyl. More preferably still, R⁰ is selected from the group consisting of t-butyl, benzyl, p-methoxybenzyl and 9-fluorenylmethyl.

As used herein, unless otherwise noted, “halogen” shall mean chlorine, bromine, fluorine and iodine.

As used herein, unless otherwise noted, the term “alkyl” whether used alone or as part of a substituent group, includes straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like.

As used herein, unless otherwise noted, “aryl” shall refer to unsubstituted carbocylic aromatic groups such as phenyl, naphthyl, and the like, preferably phenyl.

As used herein, unless otherwise noted, “aralkyl” shall mean any lower alkyl group substituted with an aryl group such as phenyl, naphthyl and the like. For example, benzyl, phenylethyl, phenylpropyl, naphthylmethyl, and the like, preferably benzyl.

As used herein, unless otherwise noted, the term “cycloalkyl” shall mean any stable monocyclic, bicyclic or polycyclic, saturated ring system, preferably a monocyclic or bicyclic saturated ring system, more preferably a monocyclic saturated ring system. Suitable examples include, but are not limited to, cyclopropyl, cyclobutyl, cyclopentyl, cyclohexyl, cycloheptyl, cyclooctyl, adamantyl, and the like.

As used herein, the notation “*” shall denote the presence of a stereogenic center.

As used herein, unless otherwise noted, the term “enantiomerically enriched” when used to describe a compound with one stereogenic center, shall mean that one stereo-configuration of the compound is present in a greater amount than the opposite stereo-configuration of said compound. Preferably, when the compound is said to be enantiomerically enriched, the desired enantiomer of said compound is present in an enantiomeric excess of at least about 75 percent ee, more preferably at least 85 percent ee, more preferably at least 90 percent ee, more preferably at least 95 percent ee, more preferably at least 98 percent ee, most preferably at least 99 percent ee.

As used herein, unless otherwise noted, the term “nitrogen protecting group” shall mean any ester group which can act as a protecting group for a amine, amide, sulfamide or sulfanomide nitrogen. Suitable examples include, but are not limited to alkoxycarbonyl, aryloxycarbonyl, aralkyloxycarbonyl, and the like. For example, the nitrogen protecting group may be of the formula —C(O)OR⁰, wherein R⁰ is C₁₋₄alkyl (preferably t-butyl), benzyl, p-methoxybenzyl, phenylethyl, phenyl, naphthyl, cycloalkyl, 9-fluorenylmethyl, and the like, and wherein any of the R⁰ groups may be further substituted.

Abbreviations used in the specification, particularly the Schemes and Examples, are as follows:

| | |
|---|---|
| BOC or Boc = | t-Butoxycarbonyl (—C(O)O—C(CH₃)₃) |
| DIPEA or DIEA = | Diisopropylethylamine |
| DMAP = | 4-(N,N-Dimethylamino)pyridine |
| DMF = | N,N-Dimethylformamide |
| EtOAc = | Ethyl acetate |
| Fmoc = | 9-Fluorenylmethoxycarbonyl |
| MTBE = | Methyl t-butyl ether |
| Pd/C = | Palladium on Carbon Catalyst |
| RBF = | Round bottom flask |
| RT or rt = | Room temperature |
| TEA = | Triethylamine |
| TFA = | Trifluoroacetic Acid |
| THF = | Tetrahydrofuran |
| XRD = | X-ray Diffraction |

As used herein, unless otherwise noted, the terms “epilepsy and related disorders” or “epilepsy or related disorder” shall mean any disorder in which a subject (preferably a human adult, child or infant) experiences one or more seizures and/or tremors. Suitable examples include, but are not limited to, epilepsy (including, but not limited to, localization-related epilepsies, generalized epilepsies, epilepsies with both generalized and local seizures, and the like), seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, and the like), essential tremor, restless limb syndrome, and the like. Preferably, the disorder is selected from epilepsy (regardless of type, underlying cause or origin), essential tremor or restless limb syndrome, more preferably, the disorder is epilepsy (regardless of type, underlying cause or origin) or essential tremor.

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) in the specification and claims are performed under suitable conditions (e.g. temperature, pressure, with appropriate solvents and/or reactants), according to known methods, to provide the desired product. The term “suitable conditions” shall mean a reaction step is performed under appropriate conditions (e.g. temperature, pressure, with appropriate solvents and/or reactants) according to known methods to provide the desired product.

One skilled in the art will also recognize that, in the specification and claims as presented herein, wherein a reagent or reagent class/type/(e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “aprotic solvent” shall mean any solvent that does not yield a proton. Suitable examples include, but are not limited to DMF, dioxane, THF, acetonitrile, pyridine, dichloroethane, dichloromethane, MTBE, toluene, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, p-methoxybenzyl, 9-fluorenylmethyl, and the like, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 75%, more preferably, the enantiomer is present at an enantiomeric excess of greater than or equal to about 85%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 75%, more preferably, the diastereomer is present at an diastereomeric excess of greater than or equal to about 85%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable base (preferably a strong base) such as NaOH, KOH, NaH, chloline hydroxide, and the like.

The present invention is directed to a process for the preparation of compounds of formula (I). Compounds of formula (IA) (compounds of formula (I) wherein R¹ is hydrogen) may be prepared as outlined in more detail in Scheme 1, below.

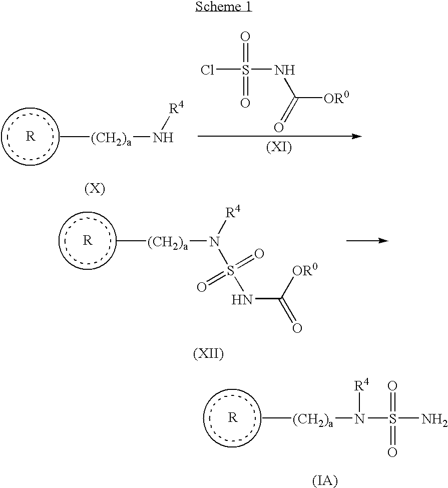

Accordingly, a suitably substituted compound of formula (X), a known compound or compound prepared by known methods, is reacted with a suitably substituted compound of formula (XI), wherein —C(O)OR⁰ is a suitably selected nitrogen protecting group, for example, an alkoxycarbonyl, aryloxycarbonyl, aralkyloxycarbonyl, and the like, for example, wherein R⁰ is C₁₋₄alkyl (preferably t-butyl), benzyl, p-methoxybenzyl, phenylethyl, phenyl, naphthyl, cycloalkyl, 9-fluorenylmethyl, and the like, and wherein any of the R⁰ groups may be further substituted, a known compound or compound prepared by known methods;

in the presence of an organic or inorganic base, wherein the organic or inorganic base is not reactive with the chloro group on the compound of formula (XI), preferably, an organic base, more preferably a tertiary amine base such DIPEA, TEA, pyridine, N-methylmorpholine, N-methylpiperidine, and the like, preferably pyridine; wherein the base is preferably present in an amount greater than about 1 molar equivalent, more preferably, the base is present in an amount in the range of from about 1.1 to about 3.0 molar equivalents; most preferably in an amount of about 2.0 molar equivalents; in an aprotic organic solvent such as DMF, THF, acetonitrile, and the like, preferably aceonitrile; preferably at a temperature in the range of from about 0° C. to about 50° C.; to yield the corresponding compound of formula (XII).

The compound of formula (XII) is de-protected according to known methods, to yield the corresponding compound of formula (IA). For example, by reacting the compound of formula (XII) with an acid or reacting the compound of formula (XII) with hydrogen or a source of hydrogen. Preferably, wherein the nitrogen protecting group (—C(O)OR⁰) is BOC, the compound of formula (XII) is de-protected by reacting with an acid such as TFA, HCl, and the like, preferably HCl; in an organic solvent such as THF, ethyl acetate, and the like. Alternatively, wherein the nitrogen protecting group (—C(O)OR⁰) is benzyl, the compound of formula (XII) is de-protected by reacting with hydrogen or a source of hydrogen, such as hydrogen gas, in the present of a catalyst such as Pd/C, at a pressure in the range of from about 10 psi to about 60 psi, in an organic solvent such as ethanol, methanol, toluene acetic acid, and the like. Alternatively, wherein the nitrogen protecting group (—C(O)OR⁰) is 9-fluorenylmethyl, the compound of formula (XII) is de-protected by reacting with a base such as an amine base such as piperidine, morpholine, and the like, in an organic solvent DMF, and the like.

Compounds of formula (IB) (compounds of formula (I) wherein R¹ is selected from the group consisting of lower alkyl) may be prepared according to the process as outlined in Scheme 2, below.

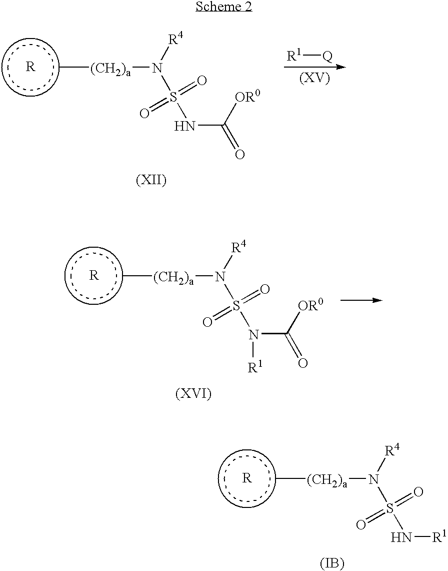

Accordingly, a suitably substituted compound of formula (XII), prepared as in Scheme 1 above, is reacting with a suitably selected compound of formula (XV), an alkyl halide or alkyl sulfonate, wherein Q is a suitable leaving group such as Cl, Br, I, —O—SO₂—CH₃ (mesylate) —O—SO₂—CF₃ (triflate)-O—SO₂-tolyl (tosylate), and the like; in the presence of an organic or inorganic base such as K₂CO₃, Na₂CO₃, NaOH, KOH, pyridine, DIPEA, TEA, and the like, preferably a tertiary amine base, more preferably pyridine; in an organic solvent such as THF, acetonitrile, DMF, and the like; preferably at a temperature in the range of from about 0° C. to about 50° C.; to yield the corresponding compound of formula (XVI).

The compound of formula (XVI) is de-protected according to known methods, to yield the corresponding compound of formula (IB). For example, by reacting the compound of formula (XVI) with an acid or reacting the compound of formula (XVI) with hydrogen or a source of hydrogen. Preferably, wherein the nitrogen protecting group (—C(O)OR⁰) is BOC, the compound of formula (XVI) is de-protected by reacting with an acid such as TFA, HCl, and the like, preferably HCl; in an organic solvent such as THF, ethyl acetate, and the like. Alternatively, wherein the nitrogen protecting group (—C(O)OR⁰) is benzyl, the compound of formula (XVI) is de-protected by reacting with hydrogen or a source of hydrogen, such as hydrogen gas, in the present of a catalyst such as Pd/C, at a pressure in the range of from about 10 psi to about 60 psi, in an organic solvent such as ethanol, methanol, toluene acetic acid, and the like. Alternatively, wherein the nitrogen protecting group (—C(O)OR⁰) is 9-fluorenylmethyl, the compound of formula (XVI) is de-protected by reacting with a base such as an amine base such as piperidine, morpholine, and the like, in an organic solvent DMF, and the like.

The compound of formula (I) is isolated according to known methods, for example by filtration, solvent evaporation, and the like. Preferably, the compound of formula (I) is further purified according to known methods, for example by recrystallization from a suitable solvent such as water, toluene, and the like, preferably toluene.

In an embodiment, the present invention is directed to a process for the preparation of the compound of formula (I-S), as outlined in Scheme 3, below.

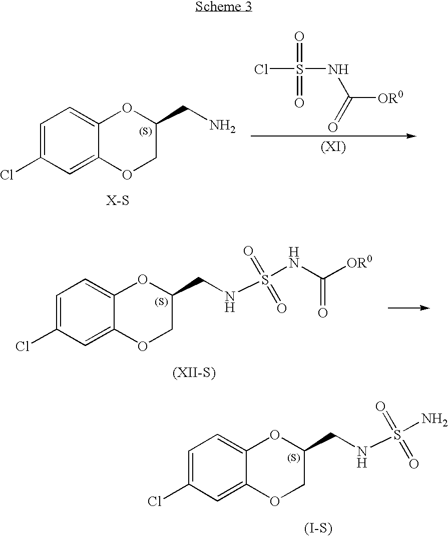

Accordingly, a suitably substituted compound of formula (X-S), also known as C-(6-chloro-2,3-dihydro-benzo[1,4]dioxin-2-yl)-methylamine, a known compound, wherein —C(O)OR⁰ is a suitably selected nitrogen protecting group, for example, an alkoxycarbonyl, aryloxycarbonyl, aralkyloxycarbonyl, and the like, for example, wherein R⁰ is C₁₋₄alkyl (preferably t-butyl), benzyl, p-methoxybenzyl, phenylethyl, phenyl, naphthyl, cycloalkyl, 9-fluorenylmethyl, and the like, and wherein any of the R⁰ groups may be further substituted, a known compound or compound prepared by known methods;

in the presence of an organic or inorganic base, wherein the organic or inorganic base is not reactive with the chloro group on the compound of formula (XI), preferably, an organic base, more preferably a tertiary amine base such DIPEA, TEA, pyridine, N-methylmorpholine, N-methylpiperidine, and the like, preferably pyridine; wherein the base is preferably present in an amount greater than about 1 molar equivalent, more preferably, the base is present in an amount in the range of from about 1.1 to about 3.0 molar equivalents; most preferably in an amount of about 2.0 molar equivalents; in an aprotic organic solvent such as DMF, THF, acetonitrile, and the like, preferably aceonitrile; preferably at a temperature in the range of from about 0° C. to about 50° C.; to yield the corresponding compound of formula (XII-S).

The compound of formula (XII-S) is de-protected according to known methods, to yield the corresponding compound of formula (I-S). For example, by reacting the compound of formula (XII-S) with an acid or reacting the compound of formula (XII-S) with hydrogen or a source of hydrogen. Preferably, wherein the nitrogen protecting group (—C(O)OR⁰) is BOC, the compound of formula (XII-S) is de-protected by reacting with an acid such as TFA, HCl, and the like, preferably HCl; in an organic solvent such as THF, ethyl acetate, and the like. Alternatively, wherein the nitrogen protecting group (—C(O)OR⁰) is benzyl, the compound of formula (XII-S) is de-protected by reacting with hydrogen or a source of hydrogen, such as hydrogen gas, in the present of a catalyst such as Pd/C, at a pressure in the range of from about 10 psi to about 60 psi, in an organic solvent such as ethanol, methanol, toluene acetic acid, and the like. Alternatively, wherein the nitrogen protecting group (—C(O)OR⁰) is 9-fluorenylmethyl, the compound of formula (XII-S) is de-protected by reacting with a base such as an amine base such as piperidine, morpholine, and the like, in an organic solvent DMF, and the like.

The compound of formula (I-S) is isolated according to known methods, for example by filtration. Preferably, the compound of formula (I-S) is further purified according to known methods, for example by recrystallization from a suitable solvent such as water, toluene, and the like, preferably toluene.

Compounds of formula (XI) are known compounds or compounds which may be prepared according to known methods. For example, the compounds of formula (XI) may be prepared according to the process as outlined in Scheme 4, below.

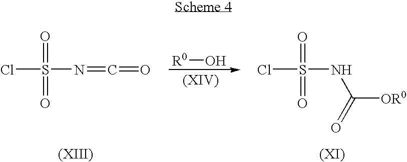

Accordingly, a suitably substituted compound of formula (XIII), a known compound or compound prepared by known methods, is reacted with a suitably selected alcohol, a compound of formula (XIV); wherein the compound of formula (XIV) is preferably present in about 1 molar equivalent; neat or in an aprotic organic solvent such as acetonitrile, ethyl acetate, toluene, and the like, provided that the compound of formula (XIII) and the compound of formula (XIV) are at least partially soluble in the solvent and unreactive to the solvent; preferably, at a temperature in the range of from about 0° to about room temperature; to yield the corresponding compound of formula (XI).

Compounds of formula (XI-S) wherein R⁰ is t-butyl may be prepared according to the process outlined in Scheme 5.

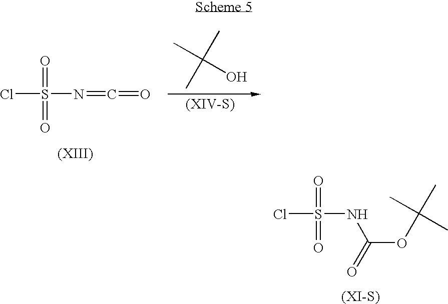

Accordingly, isocyanatidosulfuryl chloride, a known compound, is reacted t-butanol, a known compound, wherein the t-butanol is preferably present in about 1 molar equivalent; neat or in an aprotic organic solvent such as, aceotnitrile, ethyl acetate, toluene, and the like, provided that the compound of formula (XIII) and the compound of formula (XIV) are at least partially soluble in the solvent and unreactive to the solvent, preferably neat or in acetonitrile; preferably, at a temperature in the range of from about 0° to about room temperature, more preferably, at a temperature of about 0° C.; to yield the corresponding compound of formula (XI-S), also known as [(1,1-dimethylethoxy)carbonyl]-sulfamoyl chloride.

The present invention is further directed to a crystalline form of the compound of formula (I-S). The crystalline form of the compound of formula (I-S) may be characterized by their corresponding Powder X-ray Diffraction (PXRD) spectra.

In an embodiment, the crystalline form of the compound of formula (I-S) may be characterized by its corresponding PXRD peaks, wherein the peaks have a relative intensity of greater than or equal to about 10% relative intensity; preferably, wherein the peaks have a relative intensity of greater than or equal to about 25% relative intensity.

In an embodiment, the crystalline form of the compound of formula (I-S) may be characterized by its corresponding PXRD peaks, wherein the peaks are defined by their position (°2θ0), d-spacing (Å) and relative intensity (%). In another embodiment, the crystalline form of the compound of formula (I-S) may be characterized by its corresponding PXRD peaks, wherein the peaks are defined by their position (°2θ) and d-spacing (Å).

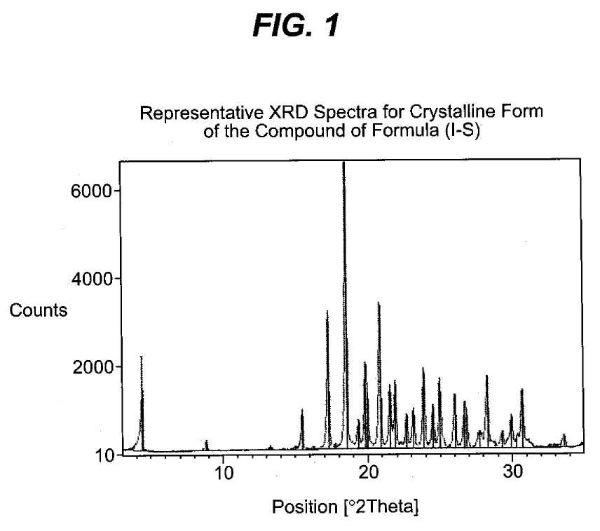

A powder XRD spectra was measured for a representative sample of the crystalline form (I-SA) of the compound of formula (I-S), as shown in FIG. 1, with characteristic peaks as listed in Table 2 below. The PXRD spectra were measured using an X-Celerator detector, scanning form 3 to 35°2θ, at a step size of 0.0165°2θ, a time per step of 10.16 sec, an effective scan speed of 0.2067°/sec, instrument voltage of 45 kV and a current setting of 40 mA.

TABLE 2
Form (I-SA)

| Position (°2θ) | d-spacing (Å) | Relative Intensity (%) |
|---|---|---|
| 4.44 | 19.92 | 33 |
| 15.50 | 5.72 | 14 |
| 17.32 | 5.12 | 48 |
| 18.57 | 4.78 | 100 |
| 19.39 | 4.58 | 10 |
| 19.86 | 4.47 | 30 |
| 20.03 | 4.43 | 20 |
| 20.88 | 4.26 | 51 |
| 21.57 | 4.12 | 23 |
| 21.93 | 4.05 | 24 |
| 22.71 | 3.92 | 13 |
| 23.19 | 3.84 | 14 |
| 23.90 | 3.72 | 29 |
| 24.53 | 3.63 | 16 |
| 25.02 | 3.56 | 25 |
| 26.04 | 3.42 | 19 |
| 26.71 | 3.34 | 16 |
| 26.84 | 3.32 | 13 |
| 28.28 | 3.16 | 26 |
| 29.96 | 2.98 | 12 |
| 30.70 | 2.91 | 21 |

The present invention further comprises pharmaceutical compositions containing one or more of the compounds prepared according to any of the processes described herein with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 1-1000 mg and may be given at a dosage of from about 0.01-300 mg/kg/day, or any range therein, preferably from about 0.5-100 mg/kg/day, or any range therein, more preferably from about 1.0-25.0 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating epilepsy or a related disorder described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of epilepsy or related disorders is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 500.0 mg/kg of body weight per day, or any range therein. Preferably, the range is from about 0.5 to about 100.0 mg/kg of body weight per day, or any range therein, more preferably, from about 1.0 to about 50.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### EXAMPLE 1

### Toluene-4-sulfonic acid 6-chloro-2,3-dihydro-benzo[1,4]dioxin-2-ylmethyl ester

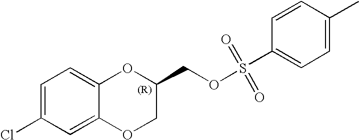

A 5 L three-necked-round bottomed flask equipped with a reflux condenser, a nitrogen outlet, an overhead stirrer, a heating mantle and a temperature control unit was charged with 6-chloro-2,3-dihydro-benzo[1,4]dioxin-2-yl)-methanol (176 g 877 mmol), tetrahydrofuran (2 L), 4-(N,N-dimethylamino)pyridine, DMAP (25 g; 204.6 mmol) and p-toluenesulfonyl chloride (200.70 g, 1.05 mol). The reaction mixture was then warmed to 40° C. An additional portion of DMAP (5 g) and p-toluenesulfonyl chloride (10 g; 52.45 mmol) were added and the reaction was continued overnight. Upon consumption of starting material the reaction was quenched with water (500 mL) and the resulting mixture extracted with MTBE (1.2 L), washed with water (300 mL, 200 mL), then with 1 N HCl (400 mL, 100 mL), and an additional water wash (100 mL), followed by a bicarbonate wash (100 mL). The organic layer was dried over Na₂SO₄ (50 g) and silica gel (60 g). The resulting solution was filtered and concentrated to yield the title compound as a thick oil.

### EXAMPLE 2

### 2-(6-Chloro-2,3-dihydro-benzo[1,4]dioxin-2-ylmethyl)-isoindole-1,3-dione

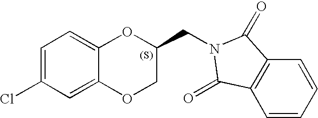

A three-necked-round bottomed flask equipped with a nitrogen outlet, a magnetic stir bar, a heating mantle and a temperature control unit was charged with 6-chloro-2,3-dihydro-benzo[1,4]dioxin-2-yl methyl ester 4-toluenesulfonate (300 g; 845.5 mmol), N,N-dimethylformamide (330.00 mL) and potassium phthalimide (203.59 g, 1.10 mol). The resulting slurry was heated to 100° C. and stirred at this temperature for 2 h. The reaction mixture was then cooled to room temperature in an ice bath and poured into a stirred ice/water mixture (1 L) with stirring. A white solid developed upon stirring. After 2 h at room temperature, the solid was collected by filteration and air dried to yield the title compound as an off-white solid.

### EXAMPLE 3

### (S)-(6-Chloro-2,3-dihydro-benzo[1,4]dioxin-2-yl)-methylamine

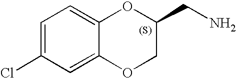

A 5 L four-necked round bottomed flask equipped with a nitrogen outlet, a reflux condenser, an overhead stirrer, a heating mantle and a temperature control unit was charged with (S)-2-((6-chloro-2,3-dihydrobenzo[b][1,4]dioxin-2-yl)methyl)isoindoline-1,3-dione (242 g; 733.9 mmol), ethanol (2.6 L) and hydrazine (48 g; 1.50 moles). The resulting mixture was heated to reflux. After 7 h the reaction mixture was cooled to room temperature and treated with HCl (2N) until acidic; and the resulting solid was filtered off. The filtrate was concentrated, then treated with MTBE (1 L) and sodium hydroxide (3N, 200 mL) and the resulting mixture stirred. The organic layer was separated and washed with water (200 mL), followed by a brine (200 mL) wash. The organic layer was dried (Na₂SO₄), filtered and concentrated to yield the title compound.

### EXAMPLE 4

### N-[[(2S)-6-chloro-2,3-dihydro-1,4-benzodioxin-2-yl]methyl]-N′-[(1,1-dimethylethoxy)carbonyl]-sulfamide

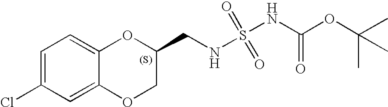

A 5 L three-necked-RBF equipped with a nitrogen outlet, a magnetic stir bar, an addition funnel, an internal thermometer and cooling bath was treated with (S)-(6-chloro-2,3-dihydro-benzo[1,4]dioxin-2-yl)-methylamine (135 g; 676.2 mmol), acetonitrile (1000 mL), 4-(N,N-dimethylamino)pyridine, DMAP (2.07 g, 16.9 mmol) and pyridine (82 mL, 80.24 g). The reaction mixture was cooled to 0° C. and a solution of Cl—SO₂—NH-Boc (168 g; 779 mmol) in acetonitrile (50 mL) was added via addition funnel. The resulting mixture was then allowed to warm to room temperature with subsequent heating to 45° C. (5 h). The reaction was then quenched by addition of 1N HCl until acidic. The product was extracted with ethyl acetate (500 mL). After phase separation, the organic layer was treated with 3N NaOH (300 mL), the basic aqueous layer was then cooled in an ice bath and treated with water (20 mL) and 2N HCl. The product was extracted with ethyl acetate (500 mL), dried (Na₂SO₄), filtered and concentrated to yield the title compound as a light yellow solid.

### EXAMPLE 5

### N-[[(2S)-6-chloro-2,3-dihydro-1,4-benzodioxin-2-yl]methyl]-sulfamide

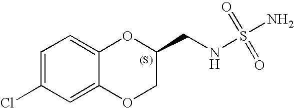

A 2 L three-necked round bottomed flask equipped with and a 250 mL addition funnel, a nitrogen outlet, an overhead stirrer, a heating mantle and a temperature control unit was charged with (S)-tert-butyl N-((6-chloro-2,3-dihydrobenzo[b][1,4]dioxin-2-yl)methyl)sulfamoylcarbamate (256 g; 540.6 mmol), ethyl Acetate (409 mL) followed by the addition of concentrated HCl (177.54 mL, 2.16 moles). The resulting mixture was warmed to 50° C. for 1 h, cooled to room temperature then neutralized by addition of NaOH (2N, 220 mL) and the product was extracted with ethyl acetate (2×200 mL), the organic layer was washed with brine (100 mL) and dried with Na₂SO₄ (20 g), filtered and concentrated to yield a thick oil (100 g crude).

The oil was taken up in hot (99-100° C.) water (950 mL). The resulting solution was hot filtered to remove insoluble oil and other impurities. The resulting mixture was then cooled to 25° C. and the resulting solid was collected by filteration and dried to yield the title compound as a white solid.

### EXAMPLE 6

### [(1,1-dimethylethoxy)carbonyl]-sulfamoyl chloride

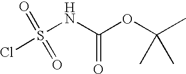

A 100 mL three-necked-RBF equipped with a nitrogen outlet, an addition funnel, a temperature sensor and an ice bath was charged with chlorosulfonyl Isocyanate (70.76 mL, 811.47 mmol) and acetonitrile (50 mL). The resulting solution was cooled to 0° C. and then t-butyl alcohol (60.15 g, 77.08 mL, 811.47 mmol) was added as a solution in acetonitile (50 mL) via the addition funnel, at such a slow rate to maintain the internal temperature of the reaction mixture at less than 10° C. At the end of the addition, the reaction was determined to be complete. The reaction mixture was concentrated to yield a white amorphous solid which was mixed with heptane and collected by filtration, then washed with heptane on the filter pad to yield the title compound.

### EXAMPLE 7

### Toluene Recrystallization of N-[[(2S)-6-chloro-2,3-dihydro-1,4-benzodioxin-2-yl]methyl]-sulfamide

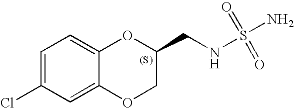

N-[[(2S)-6-chloro-2,3-dihydro-1,4-benzodioxin-2-yl]methyl]sulfamide (0.5 g) was dissolved in hot toluene (5 mL), hot-filtered to remove insoluble material and then allowed to slowly cool to room temperature. The resulting solid was collected by filtration, washed with heptane and air dried to yield the title compound as a crystalline, white solid.

m.p. 98° C.

### EXAMPLE 8

### Liquid Formulation

The compound of formula (I-S), prepared for example, as in Example 7 above, was formulated according to known methods into liquid formulations of 25 mg and 100 mg, respectively, with components as listed in Table 3 below.

TABLE 3
Liquid Formulations

| | | 25 mg/mL | 100 mg/mL |
|---|---|---|---|
| Component | Role | Suspension | Suspension |
| Compound of formula (I-S) | Active | 25 mg | 100 mg |
| Hypromellose | Suspending | 5 mg | 5 mg |
| (also known as HPMC or | agent | | |
| hydroxypropylmethyl- | | | |
| cellulose) | | | |
| Purified water | Solvent | q.s. ad. 1 mL | q.s. ad. 1 mL |

### EXAMPLE 9

### Prophetic Example

As a specific embodiment of an oral composition, 100 mg of the compound of formula (I-S), prepared as in Example 7 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (IA) 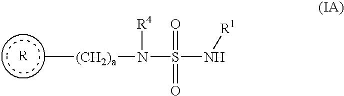
wherein
R¹ is hydrogen;
R⁴ is selected from the group consisting of hydrogen and lower alkyl;
a is an integer from 1 to 2; 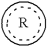
is selected from the group consisting of 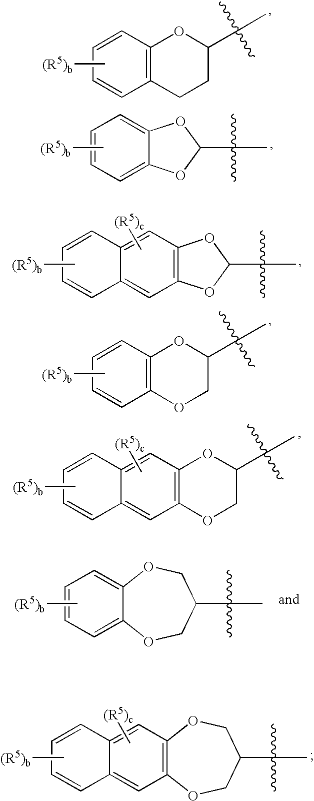
wherein b is an integer from 0 to 4; and wherein c is an integer from 0 to 2;
each R⁵ is independently selected from the group consisting of halogen, lower alkyl and nitro;
provided that when 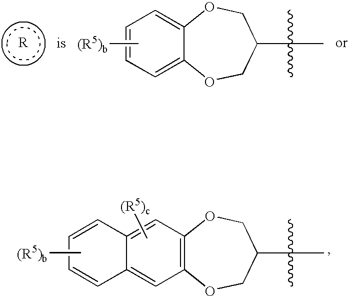
then a is 1;
or a pharmaceutically acceptable salt thereof;
comprising 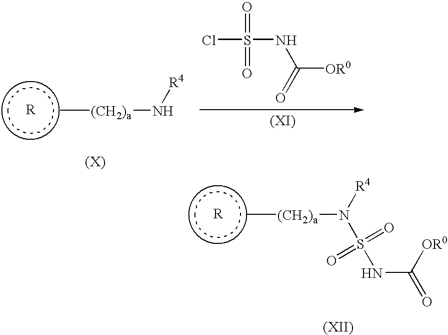
reacting a compound of formula (X) with a compound of formula (XI) wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII); 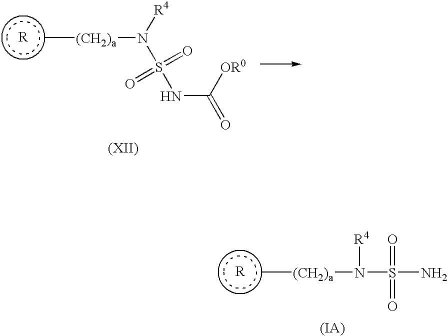
de-protecting the compound of formula (XII); to yield the corresponding compound of formula (IA).

2. A process as in claim 1, wherein a is 1; R⁴ is hydrogen and 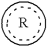
is 2-(6-chloro-2,3-dihydro-benzo[1,4]dioxinyl).

3. A process as in claim 1, wherein the organic or inorganic base is a tertiary amine base selected from the group consisting of DIPEA, TEA, pyridine, N-methylmorpholine and N-methylpiperidine.

4. A process as in claim 1, wherein the organic or inorganic base is pyridine.

5. A process as in claim 3, wherein the tertiary amine base is present in an amount in the range of from about 1.1 to about 3.0 molar equivalents.

6. A process as in claim 5, wherein the tertiary amine base is present in an amount of about 2.0 molar equivalents.

7. A process as in claim 1, wherein the aprotic organic solvent is selected from the group consisting of DMF, THF and acetonitrile.

8. A process as in claim 7, wherein the aprotic organic solvent is acetonitrile.

9. A process as in claim 1, wherein —C(O)OR⁰ is selected from the group consisting of C₁₋₄alkoxycarbonyl, aryloxycarbonyl and aralkyloxycarbonyl.

10. A process as in claim 9, wherein —C(O)OR⁰ is selected from the group consisting of lower alkyl, benzyl, p-methoxybenzyl and 9-fluorenylmethyl.

11. A process as in claim 1, wherein —C(O)OR⁰ is —C(O)O-t-butyl.

12. A process as in claim 1, wherein the compound of formula (XII) is de-protected by reacting the compound of formula (XII) with an acid.

13. A process as in claim 12, wherein the compound of formula (XII) is de-protected by reacting the compound of formula (XII) with hydrochloric acid.

14. A process for the preparation of a compound of formula (IB) 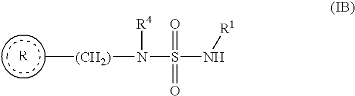
wherein
R¹ is selected from the group consisting of lower alkyl;
R⁴ is selected from the group consisting of hydrogen and lower alkyl;
a is an integer from 1 to 2; 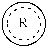
is selected from the group consisting of 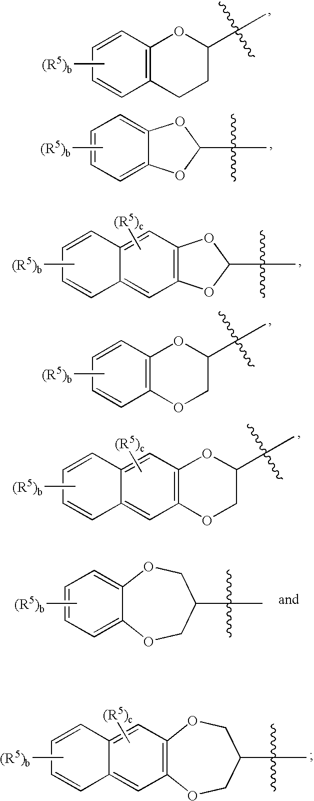
wherein b is an integer from 0 to 4; and wherein c is an integer from 0 to 2;
each R⁵ is independently selected from the group consisting of halogen, lower alkyl and nitro;
provided that when 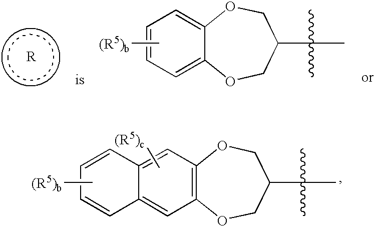
then a is 1;
or a pharmaceutically acceptable salt thereof;
comprising 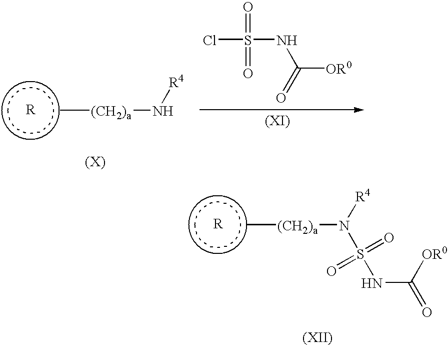
reacting a compound of formula (X) with a compound of formula (XI) wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII); 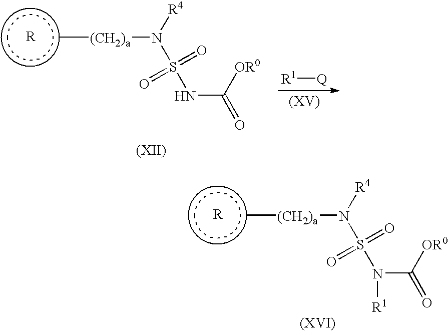
reacting the compound of formula (XII) with a compound of formula (XV), wherein Q is a leaving group; in an organic solvent; to yield the corresponding compound of formula (XVI) 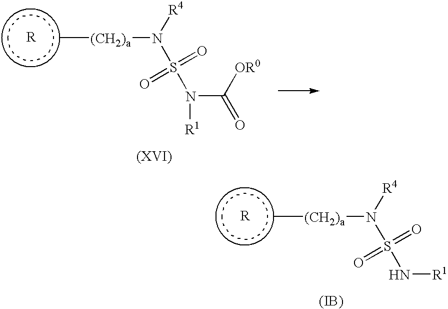
de-protecting the compound of formula (XVI); to yield the corresponding compound of formula (IB).

15. A process as in claim 14, wherein the organic or inorganic base is a tertiary amine base selected from the group consisting of DIPEA, TEA, pyridine, N-methylmorpholine and N-methylpiperidine.

16. A process as in claim 15, wherein the organic or inorganic base is pyridine.

17. A process as in claim 14, wherein the tertiary amine base is present in an amount in the range of from about 1.1 to about 3.0 molar equivalents.

18. A process as in claim 17, wherein the tertiary amine base is present in an amount of about 2.0 molar equivalents.

19. A process as in claim 14, wherein the aprotic organic solvent is selected from the group consisting of DMF, THF and acetonitrile.

20. A process as in claim 19, wherein the aprotic organic solvent is acetonitrile.

21. A process as in claim 14, wherein —C(O)OR⁰ is selected from the group consisting of C₁₋₄alkoxycarbonyl, aryloxycarbonyl and aralkyloxycarbonyl.

22. A process as in claim 21, wherein —C(O)OR⁰ is selected from the group consisting of lower alkyl, benzyl, p-methoxybenzyl and 9-fluorenylmethyl.

23. A process as in claim 14, wherein —C(O)OR⁰ is —C(O)O-t-butyl.

24. A process as in claim 14, wherein Q is selected from the group consisting of Cl, Br, I, —O—SO₂—CH₃, —O—SO₂—CF₃, —O—SO₂-tolyl.

25. A process as in claim 14, wherein the compound of formula (XII) is de-protected by reacting the compound of formula (XII) with an acid.

26. A process as in claim 25, wherein the compound of formula (XII) is de-protected by reacting the compound of formula (XII) with hydrochloric acid.

27. A process for the preparation of a compound of formula (I-S) 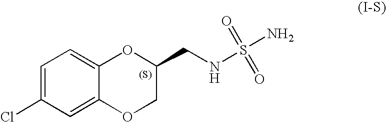
or a pharmaceutically acceptable salt thereof; comprising 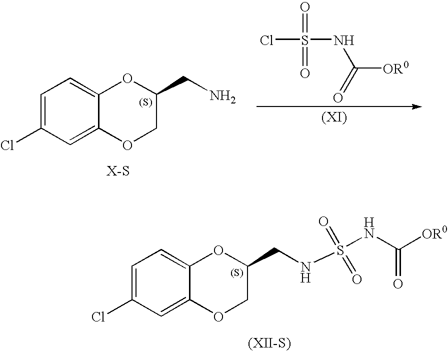
reacting a compound of formula (X-S) with a compound of formula (XI), wherein —C(O)OR⁰ is a nitrogen protecting group; in the presence of an organic or inorganic base, wherein the organic or inorganic base is not reactive with the chloro group on the compound of formula (XI); in an aprotic organic solvent; to yield the corresponding compound of formula (XII-S); 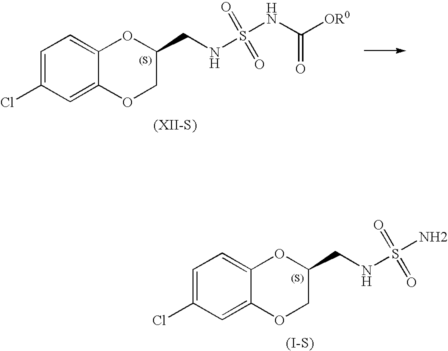
de-protecting the compound of formula (XII-S); to yield the corresponding compound of formula (I-S).

28. A process as in claim 27, wherein the organic or inorganic base is a tertiary amine base selected from the group consisting of DIPEA, TEA, pyridine, N-methylmorpholine and N-methylpiperidine.

29. A process as in claim 27, wherein the organic or inorganic base is pyridine.

30. A process as in claim 28, wherein the tertiary amine base is present in an amount in the range of from about 1.1 to about 3.0 molar equivalents.

31. A process as in claim 30, wherein the tertiary amine base is present in an amount of about 2.0 molar equivalents.

32. A process as in claim 27, wherein the aprotic organic solvent is selected from the group consisting of DMF, THF and acetonitrile.

33. A process as in claim 32, wherein the aprotic organic solvent is acetonitrile.

34. A process as in claim 27, wherein —C(O)OR⁰ is selected from the group consisting of C₁₋₄alkoxycarbonyl, aryloxycarbonyl and aralkyloxycarbonyl.

35. A process as in claim 34, wherein —C(O)OR⁰ is selected from the group consisting of lower alkyl, benzyl, p-methoxybenzyl and 9-fluorenylmethyl.

36. A process as in claim 27, wherein —C(O)OR⁰ is —C(O)O-t-butyl.

37. A process as in claim 27, wherein the compound of formula (XII-S) is de-protected by reacting the compound of formula (XII-S) with an acid.

38. A process as in claim 37, wherein the compound of formula (XII-S) is de-protected by reacting the compound of formula (XII-S) with hydrochloric acid.

39. A process as in claim 27, wherein the compound of formula (I-S) is further recrystallized.

40. A process as in claim 39, wherein the compound of formula (I-S) is recrystallized from a solvent selected from the group consisting of water and toluene.

41. A compound of formula (XII) 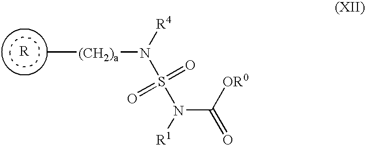
wherein
—C(O)OR⁰ is a nitrogen protecting group;
R¹ is selected from the group consisting of hydrogen and lower alkyl;
R⁴ is selected from the group consisting of hydrogen and lower alkyl;
a is an integer from 1 to 2; 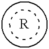
is selected from the group consisting of 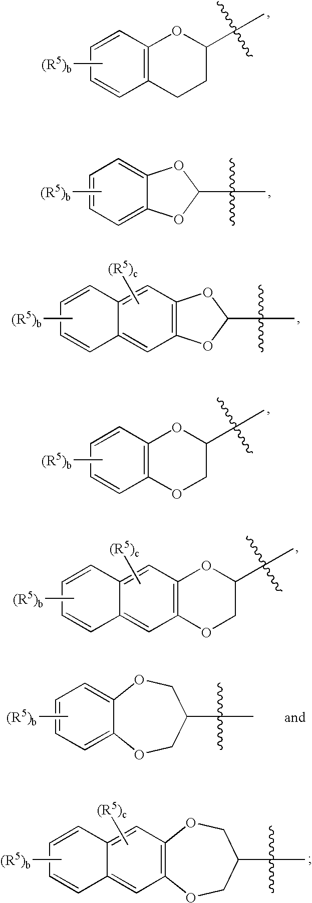
wherein b is an integer from 0 to 4; and wherein c is an integer from 0 to 2;
each R⁵ is independently selected from the group consisting of halogen, lower alkyl and nitro;
provided that when 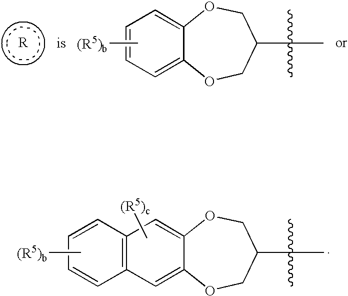
then a is 1.

42. A compound as in claim 41, wherein a is 1; R⁴ is hydrogen and 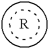
is 2-(6-chloro-2,3-dihydro-benzo[1,4]dioxinyl).

43. A compound as in claim 41, wherein —C(O)OR⁰ is selected from the group consisting of C₁₋₄alkoxycarbonyl, aryloxycarbonyl and aralkyloxycarbonyl.

44. A process as in claim 43, wherein —C(O)OR⁰ is selected from the group consisting of lower alkyl, benzyl, p-methoxybenzyl and 9-fluorenylmethyl.

45. A process as in claim 41, wherein —C(O)OR⁰ is —C(O)O-t-butyl.

46. A process as in claim 39, wherein the compound of formula (I-S) is recrystallized from toluene.

47. A compound of formula (XII-S) 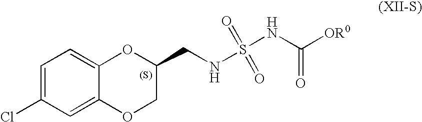
wherein —C(O)OR⁰ is a nitrogen protecting group.

48. A compound as in claim 47, wherein —C(O)OR⁰ is selected from the group consisting of C₁₋₄alkoxycarbonyl, aryloxycarbonyl and aralkyloxycarbonyl.

49. A process as in claim 47, wherein —C(O)OR⁰ is selected from the group consisting of lower alkyl, benzyl, p-methoxybenzyl and 9-fluorenylmethyl.

50. A process as in claim 47, wherein —C(O)OR⁰ is —C(O)O-t-butyl.

51. A product prepared according to the process of claim 1.

52. A product prepared according to the process of claim 27.

53. A pharmaceutical composition comprising a pharmaceutically acceptable carrier and the product of claim 52.

54. A pharmaceutical composition made by mixing the product of claim 52 and a pharmaceutically acceptable carrier.

55. A process for making a pharmaceutical composition comprising mixing the product of claim 52 and a pharmaceutically acceptable carrier.

56. A method of treating epilepsy or a related disorder comprising administering to a subject in need thereof a therapeutically effective amount of the product of claim 52.

57. A method as in claim 56, wherein the disorder is epilepsy.

58. Crystalline form (I-SA) of the compound of formula (I-S)

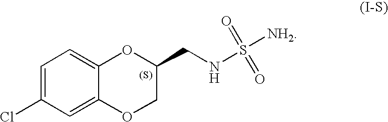

59. The crystalline form (I-SA) of claim 58 wherein crystalline form (I-SA) has the following powder X-ray diffraction peaks, as defined to position and d-spacing:

| Position (°2θ) | d-spacing (Å) |
|---|---|
| 4.44 | 19.92 |
| 15.50 | 5.72 |
| 17.32 | 5.12 |
| 18.57 | 4.78 |
| 19.39 | 4.58 |
| 19.86 | 4.47 |
| 20.03 | 4.43 |
| 20.88 | 4.26 |
| 21.57 | 4.12 |
| 21.93 | 4.05 |
| 22.71 | 3.92 |
| 23.19 | 3.84 |
| 23.90 | 3.72 |
| 24.53 | 3.63 |
| 25.02 | 3.56 |
| 26.04 | 3.42 |
| 26.71 | 3.34 |
| 26.84 | 3.32 |
| 28.28 | 3.16 |
| 29.96 | 2.98 |
| 30.70 | 2.91 |
